(19) United States
(12) Patent Application Publication
(10) Pub. No.: US 2009/0326223 A1
(43) Pub. Date: Dec. 31, 2009

(54) SYNTHESIS OF 2-AMINO-SUBSTITUTED 4-OXO-4H-CHROMEN-8.YL-TRIFLUORO-METHANESULFONIC ACID ESTERS

(76) Inventors: Roger John Griffin, Tyne and Wear (GB); Ian Robert Hardcastle, Tyne and Wear (GB); Marine Desage-El Murr, Paris (FR); Sonsoles Rodriguez-Aristegui, Madrid (ES); Bernard Thomas Golding, Tyne and Wear (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/374,354

(22) PCT Filed: Jul. 18, 2007
(86) PCT No.: PCT/GB07/02718

§ 371 Date: Feb. 27, 2009

Related U.S. Application Data
(60) Provisional application No. 60/807,651, filed on Jul. 18, 2006.

(51) Int. Cl. C07D 413/14

(52) U.S. Cl. 544/146

(57) ABSTRACT

A method of synthesising a compound of formula (I): wherein R_{N1} and R_{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms; from a compound of formula (III): comprising the steps of: (a) removing the allyl group from the compound of formula (III) with appropriate reaction conditions to yield a compound of formula (II): and (b) reacting the compound of formula (II) with a triflating agent to yield a compound of formula (I).

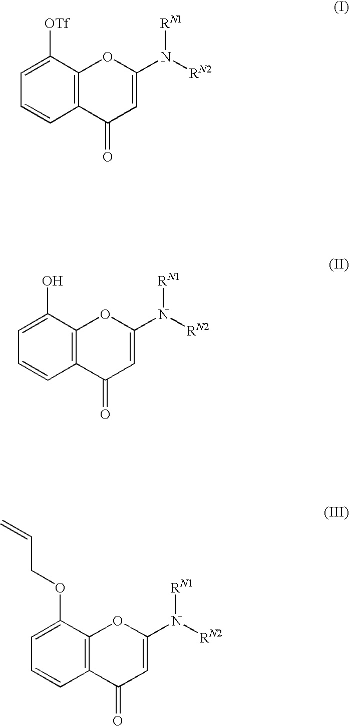

The present invention relates to improved methods of synthesis of chromenone triflates and compounds derived from them.

The following compound:

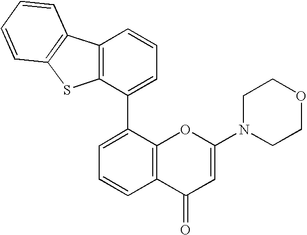

has been disclosed as inhibiting DNA-dependent protein kinase (DNA-PK) in WO 03/024949, Leahy, J. J. J., et al., Bioorg. Med. Chem. Lett., 14, 6083-6087 (2004) and Hardcastle, I. R., et al., J. Med. Chem., 48, 7829-7846 (2005).

Subsequently, derivatives of that compound which also inhibit DNA-PK have been disclosed in WO 2006/032869.

These compounds have generally been synthesised from the intermediate of formula (A):

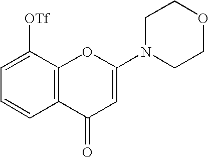

This compound was synthesised according to the following method described in WO 03/024949:

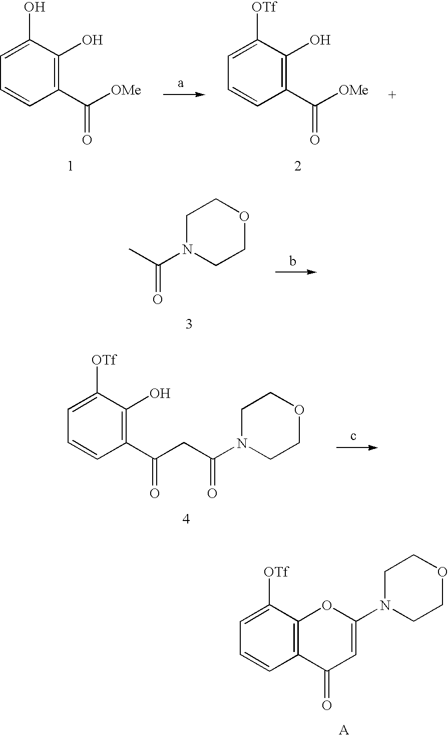

Step a: Pyridine (0.96 ml, 11.9 mmol) and dimethylaminopyridine (0.07 g, 0.58 mmol) were added to a sample of methyl 2,3-dihydroxybenzoate (1)(4.00 g, 23.80 mmol) dissolved in dichloromethane (25 ml). The mixture was cooled to 0° C. and trifluoromethane sulfonic anhydride (4.40 ml, 26.18 mmol) was added dropwise by syringe. The reaction mixture was warmed to room temperature and left to stir for 60 hours. The organic layer was washed with 1M HCl (40 ml), dried (Na₂SO₄) and concentrated to dryness in vacuo. The solid was recrystallized from ethyl acetate to yield white crystals (2)(2.62 g, 8.73 mmol, 37% yield)

Step b: A solution of diisopropylamine (5.1 ml, 3.0 mmol) in THF (30 ml) was cooled to −70° C. and slowly treated with 2.5 M solution of n-butyl lithium in hexane (14.0 ml, 35 mmol) and then warmed to 0° C. and stirred for 15 minutes. The solution was cooled to −10° C. and slowly treated with a solution of N-acetylmorpholine (3) in THF (25 ml), maintaining the temperature below −10° C. The reaction mixture was stirred at this temperature for 90 minutes and then treated with a solution of 2-hydroxy-3-trifluoromethanesulfonyloxy-benzoic acid methyl ester (2) in THF (25 ml), followed by additional THF (5 ml). The reaction mixture was slowly warmed to room temperature and stirred for 16 hours. The solution was quenched with water (5 ml) and 2 M hydrochloric acid (50 ml) and extracted into DCM (3×80 ml). The organic extracts were combined, washed with brine (50 ml), dried over sodium sulphate and evaporated in vacuo to give an oily residue. The crude product was stirred vigorously in hot ether, causing precipitation of a white solid. This was collected, after cooling in ice, by filtration and washed with cold ether, to provide the desired compound (4) as a pale brown solid (1.10 g, 2.54 mmol, 36% yield)

Step c: A solution of trifluoro-methanesulfonic acid 2-hydroxy-3-(3-morpholin-4-yl-3-oxo-propionyl)-phenyl ester (4) in DCM (35 ml) was treated with triflic anhydride (3.8 ml, 23 mmol) and stirred at room temperature under nitrogen for 16 hours. The mixture was evaporated in vacuo and then re-dissolved in methanol (80 ml). The solution was stirred for 4 hours, treated with water (80 ml) and stirred for a further hour. The mixture was evaporated in vacuo to remove methanol. The aqueous mixture was adjusted to pH 8 by treatment with saturated sodium bicarbonate and then extracted into DCM (3×150 ml). The extracts were dried over sodium sulphate and evaporated in vacuo to give a solid. The crude product was partially dissolved in DCM and loaded onto a silica column, eluting with DCM followed by (1%; 2%; 5%) methanol in DCM. All fractions containing the desired product were combined and evaporated in vacuo to give an orange solid. The crude product was dissolved in hot methanol, treated with charcoal, filtered through celite and recrystallised from methanol to provide the desired compound, trifluoro-methanesulfonic acid 2-morpholin-4-yl-4-oxo-4H-chromen-8-yl ester (A) as a white solid (0.25 g, 0.662 mmol, 28.79% yield).

The total yield of this method was 3.9% overall.

In view of the importance of the intermediate, the present inventors have devised routes to the intermediate and related compounds which have an improved yield.

Accordingly, a first aspect of the present invention provides a method of synthesising a compound of formula (I):

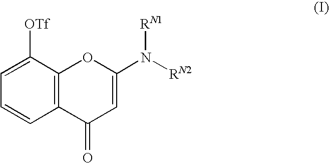

wherein R^{N1} and R^{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
from a compound of formula (III):

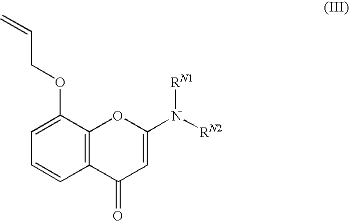

comprising the steps of:
(a) removing the allyl group from the compound of formula (III) with appropriate reaction conditions to yield a compound of formula (II):

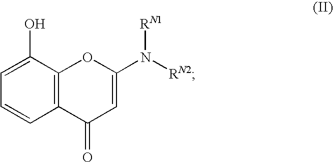

and
(b) reacting the compound of formula (II) with a triflating agent to yield a compound of formula (I).

The allyl group may be removed by any appropriate reaction conditions. Such appropriate reaction conditions are listed in pages 68 to 72 of Protective Groups in Organic Synthesis, Greene, T. W. and Wuts, P. G. M., 3^{rd} Edition, John Wiley & Sons, 1999, which is incorporated herein by reference. In particular, as with the removal of all protecting groups, the conditions should be such that the remainder of the molecule being deprotected is unaffected. In particular, removal is preferably achieved using Wilkinson's catalyst, Rh(PPh₃)₃Cl, in the presence of 1,4-diaza-bicyclo[2.2.2]octane (DABCO) in ethanol. This catalyst has been found to carry out this reaction without the need for the typical second acidic cleavage step.

The triflating step may be carried out using any known triflating agent, such as triflic anhydride or N-phenyltrifluoromethanesulfonimide (PhNTf₂). In some embodiments of the present invention, PhNTf₂ in triethylamine is used.

The compound of formula (III) can be synthesised from a compound of formula (IV):

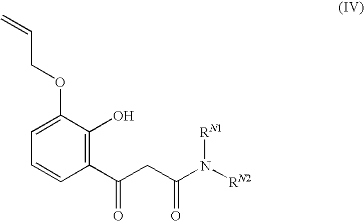

by ring closure. Accordingly, a preferred embodiment of the first aspect of the present invention further comprises ring closing a compound of formula (IV) to produce a compound of formula (III).

Ring closure of compounds of formula (IV) requires treatment with an acid anhydride, such as triflic anhydride, in a suitably compatible solvent, for example, DCM.

The compound of formula (IV) can be synthesised by two possible routes. In one set of embodiments, the method of the first aspect further comprises synthesising the compound of formula (IV) from a compound of formula (V):

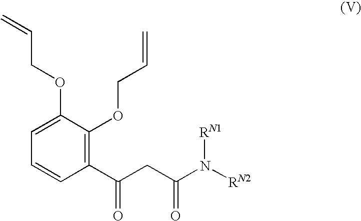

by selective removal of the 2-allyl group. Accordingly, a further preferred embodiment of the above embodiment comprises synthesising a compound of formula (IV) from a compound of formula (V) by selective removal of the 2-allyl group.

The selective removal of the 2-allyl group of a compound of formula (V) is preferably carried out using TiCl₄ and Bu₄NI.

The compound of formula (V) can be synthesised by coupling compound 7:

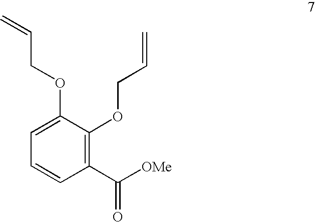

with a compound of formula (VI):

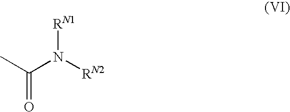

Accordingly, a preferred embodiment of the above embodiment further comprises the step of coupling compound 7 with a compound of formula (VI).

The coupling of compound 7 with a compound of formula (VI) may be achieved by generating the metal, for example lithium, enolate of the compound of formula (VI) in situ, for example by the use of metal, particularly lithium, diisopropylamide (LDA) in a suitably compatible solvent, such as THF.

Compound 7 may be made from the compound 1:

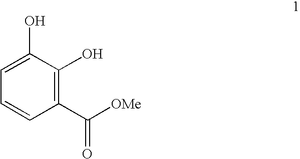

by converting both phenolic groups to allyl ether groups. Accordingly, a further preferred embodiment of the above embodiment further comprises the step of converting both phenolic groups on compound 1 to allyl ether groups to yield compound 7.

The conversion of the phenolic groups of compound 1 to yield compound 7 may be carried out by standard conditions, for example as listed in pages 67 and 86 of Protective Groups in Organic Synthesis, Greene, T. W. and Wuts, P. G. M., 3^{rd} Edition, John Wiley & Sons, 1999, which is incorporated herein by reference. In some embodiments, allyl bromide may be used, for example with base (e.g. potassium carbonate) in a suitably compatible solvent, such as acetonitrile.

In an alternative set of embodiments, the method of the first aspect further comprises synthesising the compound of formula (IV) from a compound of formula (VII):

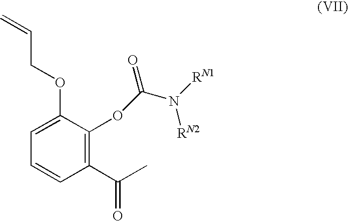

by a Baker-Venkataraman rearrangement. Accordingly, a further preferred embodiment of the first aspect of the present invention comprises synthesising a compound of formula (IV) from a compound of formula (VII) by a Baker-Venkataraman rearrangement.

The Baker-Venkataraman rearrangement may be carried out using standard reaction conditions, i.e. with the use of base. In some embodiments, potassium hydroxide in a suitably compatible solvent, such as pyridine, may be used.

The compound of formula (VII) can be synthesised by coupling compound 17:

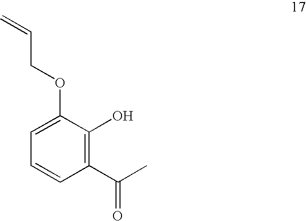

with a compound of formula (VIII):

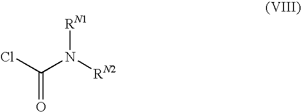

Accordingly, a further preferred embodiment of the above embodiment comprises coupling compound 17 with a compound of formula (VIII) to yield a compound of formula (VII).

The coupling of compound 17 with a compound of formula (VIII) may be achieved by using, for example, cesium carbonate in a suitably compatible solvent, such as acetonitrile.

The compound 17 can be synthesised from compound 16:

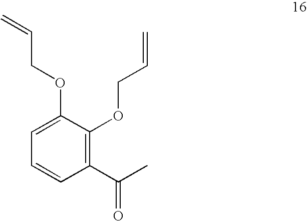

by selective removal of the 2-allyl group. Accordingly a further preferred embodiment of the above embodiment further comprises the step of selectively removing the 2-allyl group of compound 16 to yield compound 17.

The compound 16 may have its 2-allyl group selectively removed in the same manner as the compound of formula (V) above.

The compound 16 can be synthesised from compound 15:

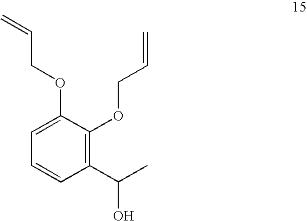

by oxidation. Accordingly a further preferred embodiment of the above embodiment further comprises the step of oxidising compound 15 to yield compound 16.

The oxidation of compound 15 may be carried out using pyridinium chlorochromate (PCC), MnO₂ or the Dess-Martin reagent, of which PCC is preferred.

The compound 15 can be synthesised from compound 14:

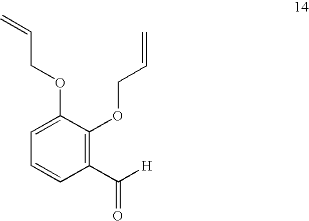

by methylation by use of a Grignard reagent. Accordingly a further preferred embodiment of the above embodiment further comprises the step of methylating compound 14 to yield compound 15.

The methylation of compound 14 may be achieved by, for example, treatment with MeMgBr.

The compound 14 can be synthesised from compound 5:

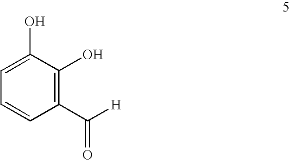

by conversion of both phenolic groups to allyl ether groups. Accordingly a further preferred embodiment of the above embodiment further comprises the step of converting both phenolic groups of compound 5 to allyl ether groups to yield compound 14.

The conversion of compound 5 may be achieved in the same way as for compound 1 described above.

The compounds of formula (I) can be used in the synthesis of compounds of formula (IX):

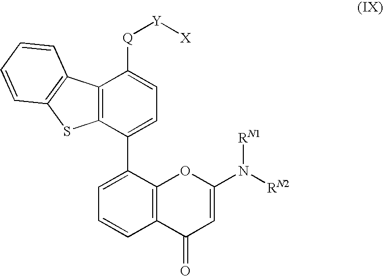

wherein:
R^{N1} and R^{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;

### Q is —NH—C(═O)— or —O—;

Y is an optionally substituted C₁₋₅ alkylene group;
X is selected from SR^{S1} or NR^{N3}R^{N4}, wherein,
R^{S1}, or R^{N3} and R^{N4} are independently selected from hydrogen, optionally substituted C₁₋₇ alkyl, C₅₋₂₀ aryl, or C₃₋₂₀ heterocyclyl groups, or R⁴ and R⁵ may together form, along with the nitrogen atom to which they are attached, an optionally substituted heterocyclic ring having from 4 to 8 ring atoms;
if Q is —O—, X is additionally selected from —C(═O)—NR^{N5}R^{N6}, wherein R^{N5} and R^{N6} are independently selected from hydrogen, optionally substituted C₁₋₇ alkyl, C₅₋₂₀ aryl, or C₃₋₂₀ heterocyclyl groups, or R^{N5} and R^{N6} may together form, along with the nitrogen atom to which they are attached, an optionally substituted heterocyclic ring having from 4 to 8 ring atoms;
and
if Q is —NH—C(═O)—, —Y—X may additionally selected from C₁₋₇ alkyl.

These compounds, and their synthesis from compounds of formula I, are described in WO 2006/032869, which is incorporated herein by reference. In general, the compounds of formula (IX) are synthesised by the Suzuki-Miyaura coupling of a precursor of the substituted dibenzothiophene group:

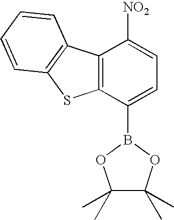

to a compound of formula I, or by conversion of the triflate to a boronate group, and then subsequent coupling of a triflate of the precursor of the substituted dibenzothiophene group.

Accordingly, a second aspect of the invention comprises the synthesis of a compound of formula (IX) from a compound of formula (I), wherein the compound of formula (I) is synthesised according to the first aspect of the invention.

Compounds of formula (I) may also be used in the synthesis of compounds of formula (X)

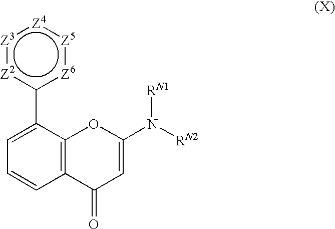

wherein:
R^{N1} and R^{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
Z², Z³, Z⁴, Z⁵ and Z⁶, together with the carbon atom to which they are bound, form an aromatic ring;
Z² is selected from the group consisting of CR², N, NH, S, and O; Z³ is CR³; Z⁴ is selected from the group consisting of CR⁴, N, NH, S, and O; Z⁵ is a direct bond, or is selected from the group consisting of O, N, NH, S, and CH; Z⁶ is selected from the group consisting of O, N, NH, S, and CH;

### R² is H;

R³ is selected from halo or optionally substituted C₅₋₂₀ aryl;
R⁴ is selected from the group consisting of H, OH, NO₂, NH₂ and Q-Y—X, where

### Q is —NH—C(═O)— or —O—;

Y is an optionally substituted C₁₋₅ alkylene group;
X is selected from SR^{S1} or NR^{N3}R^{N4}, wherein,
R^{S1}, or R^{N3} and R^{N4} are independently selected from hydrogen, optionally substituted C₁₋₇ alkyl, C₅₋₂₀ aryl, or C₃₋₂₀ heterocyclyl groups, or R^{N3} and R^{N4} may together form, along with the nitrogen atom to which they are attached, an optionally substituted heterocyclic ring having from 4 to 8 ring atoms;
if Q is —O—, X may additionally be selected from —C(═O)—NR^{N5}R^{N6}, wherein R^{N5} and R^{N6} are independently selected from hydrogen, optionally substituted C₁₋₇ alkyl, C₅₋₂₀ aryl, or C₃₋₂₀ heterocyclyl groups, or R^{N5} and R^{N6} may together form, along with the nitrogen atom to which they are attached, an optionally substituted heterocyclic ring having from 4 to 8 ring atoms and
if Q is —NH—C(═O)—, —Y—X may be additionally selected from C₁₋₇ alkyl.

Z², Z³, Z⁴, Z⁵ and Z⁶ are selected such that the group they form including the carbon atom to which Z² and Z⁶ are bound is aromatic.

These compounds, and their synthesis from compounds of formula (I) are described in co-pending applications PCT/GB2006/001379 and U.S. Ser. No. 11/403,763, which are incorporated herein by reference. In generally, the compounds of formula (X) are synthesised by the Suzuki-Miyaura coupling of a precursor of the substituted phenyl group, e.g.:

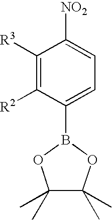

to a compound of formula I, or by conversion of the triflate to a boronate group, and then subsequent coupling of a triflate of the precursor of the substituted phenyl group.

Accordingly, a third aspect of the invention comprises the synthesis of a compound of formula (X) from a compound of formula (I), wherein the compound of formula (I) is synthesised according to the first aspect of the invention.

### DEFINITIONS

C₁₋₇ alkyl: The term “C₁₋₇ alkyl”, as used herein, pertains to a monovalent moiety obtained by removing a hydrogen atom from a C₁₋₇ hydrocarbon compound having from 1 to 7 carbon atoms, which may be aliphatic or alicyclic, or a combination thereof, and which may be saturated, partially unsaturated, or fully unsaturated.

Examples of saturated linear C₁₋₇ alkyl groups include, but are not limited to, methyl, ethyl, n-propyl, n-butyl, and n-pentyl(amyl).

Examples of saturated branched C₁₋₇ alkyl groups include, but are not limited to, iso-propyl, iso-butyl, sec-butyl, tert-butyl, and neo-pentyl.

Examples of saturated alicyclic C₁₋₇ alkyl groups (also referred to as “C₃₋₇ cycloalkyl” groups) include, but are not limited to, groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of unsaturated C₁₋₇ alkyl groups which have one or more carbon-carbon double bonds (also referred to as “C₂₋₇alkenyl” groups) include, but are not limited to, ethenyl(vinyl, —CH═CH₂), 2-propenyl(allyl, —CH—CH═CH₂), isopropenyl (—C(CH₃)═CH₂), butenyl, pentenyl, and hexenyl.

Examples of unsaturated C₁₋₇ alkyl groups which have one or more carbon-carbon triple bonds (also referred to as “C₂₋₇ alkynyl” groups) include, but are not limited to, ethynyl (ethinyl) and 2-propynyl(propargyl).

Examples of unsaturated alicyclic (carbocyclic) C₁₋₇ alkyl groups which have one or more carbon-carbon double bonds (also referred to as “C₃₋₇cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

C₃₋₂₀ heterocyclyl: The term “C₃₋₂₀ heterocyclyl”, as used herein, pertains to a monovalent moiety obtained by removing a hydrogen atom from a ring atom of a C₃₋₂₀ heterocyclic compound, said compound having one ring, or two or more rings (e.g., spiro, fused, bridged), and having from 3 to 20 ring atoms, atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms. “C₃₋₂₀” denotes ring atoms, whether carbon atoms or heteroatoms.

Examples of C₃₋₂₀ heterocyclyl groups having one nitrogen ring atom include, but are not limited to, those derived from aziridine, azetidine, pyrrolidines (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine.

Examples of C₃₋₂₀ heterocyclyl groups having one oxygen ring atom include, but are not limited to, those derived from oxirane, oxetane, oxolane (tetrahydrofuran), oxole (dihydrofuran), oxane (tetrahydropyran), dihydropyran, pyran (C₆), and oxepin. Examples of substituted C₃₋₂₀ heterocyclyl groups include sugars, in cyclic form, for example, furanoses and pyranoses, including, for example, ribose, lyxose, xylose, galactose, sucrose, fructose, and arabinose.

Examples of C₃₋₂₀ heterocyclyl groups having one sulphur ring atom include, but are not limited to, those derived from thiirane, thietane, thiolane (tetrahydrothiophene), thiane (tetrahydrothiopyran), and thiepane.

Examples of C₃₋₂₀ heterocyclyl groups having two oxygen ring atoms include, but are not limited to, those derived from dioxolane, dioxane, and dioxepane.

Examples of C₃₋₂₀ heterocyclyl groups having two nitrogen ring atoms include, but are not limited to, those derived from imidazolidine, pyrazolidine (diazolidine), imidazoline, pyrazoline (dihydropyrazole), and piperazine.

Examples of C₃₋₂₀ heterocyclyl groups having one nitrogen ring atom and one oxygen ring atom include, but are not limited to, those derived from tetrahydrooxazole, dihydrooxazole, tetrahydroisoxazole, dihydroisoxazole, morpholine, tetrahydrooxazine, dihydrooxazine, and oxazine.

Examples of C₃₋₂₀ heterocyclyl groups having one oxygen ring atom and one sulphur ring atom include, but are not limited to, those derived from oxathiolane and oxathiane (thioxane).

Examples of C₃₋₂₀ heterocyclyl groups having one nitrogen ring atom and one sulphur ring atom include, but are not limited to, those derived from thiazoline, thiazolidine, and thiomorpholine.

Other examples of C₃₋₂₀heterocyclyl groups include, but are not limited to, oxadiazine and oxathiazine.

Examples of heterocyclyl groups which additionally bear one or more oxo (═O) groups, include, but are not limited to, those derived from:

C₅ heterocyclics, such as furanone, pyrone, pyrrolidone (pyrrolidinone), pyrazolone (pyrazolinone), imidazolidone, thiazolone, and isothiazolone;
C₆ heterocyclics, such as piperidinone (piperidone), piperidinedione, piperazinone, piperazinedione, pyridazinone, and pyrimidinone (e.g., cytosine, thymine, uracil), and barbituric acid;
fused heterocyclics, such as oxindole, purinone (e.g., guanine), benzoxazolinone, benzopyrone (e.g., coumarin);
cyclic anhydrides (—C(═O)—O—C(═O)— in a ring), including but not limited to maleic anhydride, succinic anhydride, and glutaric anhydride;
cyclic carbonates (—O—C(═O)—O— in a ring), such as ethylene carbonate and 1,2-propylene carbonate;
imides (—C(═O)—NR—C(═O)— in a ring), including but not limited to, succinimide, maleimide, phthalimide, and glutarimide;
lactones (cyclic esters, —O—C(═O)— in a ring), including, but not limited to, β-propiolactone, γ-butyrolactone, δ-valerolactone (2-piperidone), and ε-caprolactone; lactams (cyclic amides, —NR—C(═O)— in a ring), including, but not limited to, β-propiolactam,
γ-butyrolactam (2-pyrrolidone), δ-valerolactam, and ε-caprolactam; cyclic carbamates (—O—C(═O)—NR— in a ring), such as 2-oxazolidone; cyclic ureas (—NR—C(═O)—NR— in a ring), such as 2-imidazolidone and pyrimidine-2,4-dione (e.g., thymine, uracil).

C₅₋₂₀ aryl: The term “C₅₋₂₀ aryl”, as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups”, in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆₋), naphthalene (C₁₀), anthracene (C₁₄), phenanthrene (C₁₄), naphthacene (C₁₈), and pyrene (C₁₆).

Examples of aryl groups which comprise fused rings, one of which is not an aromatic ring, include, but are not limited to, groups derived from indene and fluorene.

Alternatively, the ring atoms may include one or more heteroatoms, including but not limited to oxygen, nitrogen, and sulphur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

Examples of C₅₋₂₀ heterocyclic groups (some of which are C₅₋₂₀ heteroaryl groups) which comprise fused rings, include, but are not limited to, C₉ heterocyclic groups derived from benzofuran, isobenzofuran, indole, isoindole, purine (e.g., adenine, guanine), benzothiophene, benzimidazole; C₁₀ heterocyclic groups derived from quinoline, isoquinoline, benzodiazine, pyridopyridine, quinoxaline; C₁₃heterocyclic groups derived from carbazole, dibenzothiophene, dibenzofuran; C₁₄ heterocyclic groups derived from acridine, xanthene, phenoxathiin, phenazine, phenoxazine, phenothiazine.

The above C₁₋₇ alkyl, C₃₋₂₀ heterocyclyl and C₅₋₂₀ aryl groups whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

C₁₋₇ alkoxy: —OR, wherein R is a C₁₋₇ alkyl group. Examples of C₁₋₇ alkoxy groups include, but are not limited to, —OCH₃ (methoxy), —OCH₂CH₃ (ethoxy) and —OC(CH₃)₃ (tert-butoxy).

Oxo(keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam (2-pyrrolidone), δ-valerolactam, and ε-caprolactam.

Imino (imine): ═NR, wherein R is an imino substituent, for example, hydrogen, C₁₋₇ alkyl group, a C₃₋₂₀heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl(carbaldehyde, carboxaldehyde): —C(═O)H.

Acyl(keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy(carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl and phthalimidyl:

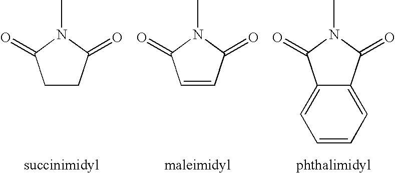

Acylureido: —N(R¹)C(O)NR²C(O)R³ wherein R¹ and R² are independently ureido substituents, for example, hydrogen, a C₁₋₇ alkyl group, a C₃₋₂₀ heterocyclyl group, or a C₅₋₂₀ aryl group, preferably hydrogen or a C₁₋₇ alkyl group. R³ is an acyl group as defined for acyl groups. Examples of acylureido groups include, but are not limited to, —NHCONHC(O)H, —NHCONMeC(O)H, —NHCONEtC(O)H, —NHCONMeC(O)Me, —NHCONEtC(O)Et, —NMeCONHC(O)Et, —NMeCONHC(O)Me, —NMeCONHC(O)Et, —NMeCONMeC(O)Me, —NMeCONEtC(O)Et, and —NMeCONHC(O)Ph.

Carbamate: —NR¹—C(O)—OR² wherein R¹ is an amino substituent as defined for amino groups and R² is an ester group as defined for ester groups. Examples of carbamate groups include, but are not limited to, —NH—C(O)—O-Me, —NMe-C(O)—O-Me, —NH—C(O)—O-Et, —NMe—C(O)—O-t-butyl, and —NH—C(O)—O-Ph.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Tetrazolyl: a five membered aromatic ring having four nitrogen atoms and one carbon atom,

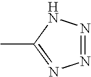

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group.

Amidine: —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. An example of an amidine group is —C(═NH)NH₂.

Carbazoyl(hydrazinocarbonyl): —C(O)—NN—R¹ wherein R¹ is an amino substituent as defined for amino groups. Examples of azino groups include, but are not limited to, —C(O)—NN—H, —C(O)—NN-Me, —C(O)—NN-Et, —C(O)—NN-Ph, and —C(O)—NN—CH₂-Ph.

Nitro: —NO₂.

Nitroso: —NO.

Azido: —N₃.

Cyano(nitrile, carbonitrile): —CN.

Isocyano: —NC.

Cyanato: —OCN.

Isocyanato: —NCO.

Thiocyano(thiocyanato): —SCN.

Isothiocyano(isothiocyanato): —NCS.

Sulfhydryl(thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃, —S(═O)₂C₄F₉ (nonaflyl), —S(═O)₂CH₂CF₃ (tresyl), —S(═O)₂Ph (phenylsulfonyl), 4-methylphenylsulfonyl(tosyl), 4-bromophenylsulfonyl(brosyl), and 4-nitrophenyl(nosyl).

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ and —OS(═O)₂CH₂CH₃.

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Sulfamyl: —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamino: —NR¹S(═O)₂R¹, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅. A special class of sulfonamino groups are those derived from sultams—in these groups one of R¹ and R is a C₅₋₂₀ aryl group, preferably phenyl, whilst the other of R¹ and R is a bidentate group which links to the C₅₋₂₀ aryl group, such as a bidentate group derived from a C₁₋₇ alkyl group. Examples of such groups include, but are not limited to:

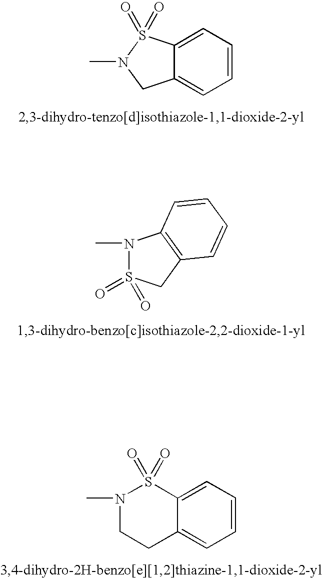

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

In many cases, substituents may themselves be substituted. For example, a C₁₋₇ alkoxy group may be substituted with, for example, a C₁₋₇ alkyl (also referred to as a C₁₋₇ alkyl-C₁₋₇alkoxy group), for example, cyclohexylmethoxy, a C₃₋₂₀ heterocyclyl group (also referred to as a C₅₋₂₀ aryl-C₁₋₇ alkoxy group), for example phthalimidoethoxy, or a C₅₋₂₀ aryl group (also referred to as a C₅₋₂₀ aryl-C₁₋₇alkoxy group), for example, benzyloxy.

### Isomers, Salts and Solvates

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

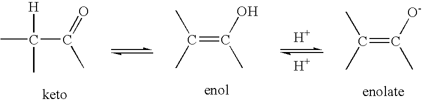

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt and solvate forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts”, J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

### Further Preferences

### All Compounds

In the present invention, it is preferred that R^{N1} and R^{N2} form, along with the nitrogen atom to which they are attached, a heterocyclic ring having from 4 to 8 atoms. This heterocyclic ring may form part of a C₄₋₂₀ heterocyclyl group defined above (except with a minimum of 4 ring atoms), which must contain at least one nitrogen ring atom. It is preferred that R^{N1} and R^{N2} form, along with the nitrogen atom to which they are attached, a heterocyclic ring having 5, 6 or 7 atoms, more preferably 6 ring atoms.

Single rings having one nitrogen atom include azetidine, azetidine, pyrrolidine (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine; two nitrogen atoms include imidazolidine, pyrazolidine (diazolidine), imidazoline, pyrazoline (dihydropyrazole), and piperazine; one nitrogen and one oxygen include tetrahydrooxazole, dihydrooxazole, tetrahydroisoxazole, dihydroisoxazole, morpholine, tetrahydrooxazine, dihydrooxazine, and oxazine; one nitrogen and one sulphur include thiazoline, thiazolidine, and thiomorpholine.

Preferred rings are those containing one heteroatom in addition to the nitrogen, and in particular, the preferred heteroatoms are oxygen and sulphur. Thus preferred groups include morpholino, thiomorpholino, thiazolinyl. Preferred groups without a further heteroatom include pyrrolidino.

The most preferred groups are morpholino and thiomorpholino.

As mentioned above, these heterocyclic groups may themselves be substituted; a preferred class of substituent is a C₁₋₇ alkyl group. When the heterocyclic group is morpholino, the substituent group or groups are preferably methyl or ethyl, and more preferably methyl. A sole methyl substituent is most preferably in the 2 position.

As well as the single ring groups listed above, rings with bridges or cross-links are also envisaged. Examples of these types of ring where the group contains a nitrogen and an oxygen atom are:

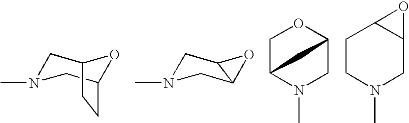

These are named 8-oxa-3-aza-bicyclo[3.2.1]oct-3-yl, 6-oxa-3-aza-bicyclo[3.1.0]hex-3-yl, 2-oxa-5-aza-bicyclo[2.2.1]hept-5-yl, and 7-oxa-3-aza-bicyclo[4.1.0]hept-3-yl, respectively.

### Compounds of Formula (IX)

When Q is —NH—C(═O)—, X is preferably NR^{N3}R^{N4}. It is further preferred that Y is an optionally substituted C₁₋₃ alkylene group, more preferably an optionally substituted C₁₋₂ alkylene group and most preferably a C₁₋₂ alkylene group.

When Q is —O— and X is NR^{N3}R^{N4}, then Y is preferably an optionally substituted C₁₋₃ alkylene group, more preferably an optionally substituted C₁₋₂ alkylene group and most preferably a C₁₋₂ alkylene group.

In some embodiments, R^{N3} and R^{N4} are preferably independently selected from H and optionally substituted C₁₋₇ alkyl, more preferably H and optionally substituted C₁₋₄ alkyl and most preferably H and optionally substituted C₁₋₂ alkyl. Preferred optional substitutents include, but are not limited to, hydroxy, methoxy, —NH₂, optionally substituted C₆ aryl and optionally substituted C₅₋₆ heterocyclyl.

In other embodiments, R^{N3} and R^{N4} form, together with the nitrogen atom to which they are attached, an optionally substituted nitrogen containing heterocylic ring having from 4 to 8 ring atoms. Preferably, the heterocyclic ring has 5 to 7 ring atoms. Examples of preferred groups include, morpholino, piperidinyl, piperazinyl, homopiperazinyl and tetrahydropyrrolo. These groups may be substituted, and a particularly preferred group is optionally substituted piperazinyl, where the substituent is preferably on the para-nitrogen atom. Preferred N-substituents include optionally substituted C₁₋₄ alkyl, optionally substituted C₆ aryl and acyl (with a C₁₋₄ alkyl group as the acyl substituent).

Some preferred compounds of the second aspect of the present invention can be represented by formula (IXa):

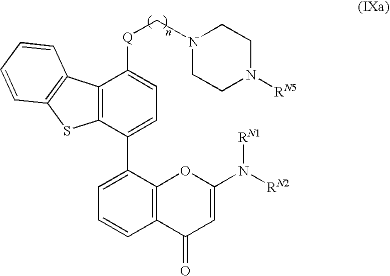

wherein:
R^{N1}, R^{N2} and Q are as defined for formula (IX);
n is 1 to 7, preferably 14 and most preferably 1 or 2; and
R^{N5} is selected from hydrogen, optionally substituted C₁₋₇ alkyl (preferably optionally substituted C₁₋₄ alkyl), optionally substituted C₅₋₂₀ aryl (preferably optionally substituted C₆ aryl), and acyl (where the acyl substituent is preferably C₁₋₄ alkyl).

The preferences for R⁶ and R⁷ may be the same as for R⁴ and R⁵ expressed above.

### Compounds of Formula (X)

### Z², Z³, Z, Z⁵ and Z¹

When Z⁵ is not a single bond, Z², Z³, Z⁴, Z⁵ and Z⁶ and the carbon atom to which Z² and Z⁶ are bound, form a six-membered aromatic ring, and it is preferred that one or two of Z², Z⁴, Z⁵ and Z⁶ are N and the rest are CH. When Z⁵ is a single bond, Z², Z³, Z⁴, Z⁵ and Z⁶ and the carbon atom to which Z² and Z⁶ are bound, form a five-membered aromatic ring, and it is preferred that one or two of Z², Z⁴ and Z⁶ are selected from S, O and N and that the rest are CH. It may be preferred that one of Z², Z⁴ and Z⁶ is selected from O and S, and that the others are both CH or one is N and the other CH.

Z², Z³, Z⁴, Z⁵ and Z⁶, together with the carbon atom to which they are bound, preferably form a substituted aryl group selected from substituted phenyl, thiophenyl, furanyl, thiazolyl, imidazolyl, pyridyl, pyrimidinyl, isoxazolyl, oxazolyl, isothiazolyl. More preferably they form a group selected from substituted phenyl, thiazolyl, thiophenyl, or pyridyl.

Z² is preferably S or CR², where R² is preferably H.

Z³ is preferably CR³. R³ is preferably optionally substituted C₅₋₂₀ aryl, more preferably C₅₋₆ aryl.

Some preferred embodiments have R³ as C₅ heteroaryl, pyridyl and phenyl, of which phenyl is most preferred. R³ is preferably unsubstituted.

In embodiments where R³ is C₅₋₂₀ aryl, it may include one or more fused rings. In these embodiments, R³ may preferably be selected from naphthyl, indolyl, quinolinyl and isoquinolinyl.

In embodiments where R³ is C₅ heteroaryl, it is preferably selected from groups derived from furan, thiophen, 2-methyl-thiophene, 2-nitrothiophene, thiophen-2-ylamine, thiazole, imidazole, and 1-methyl-1H-imidazole.

In embodiments where R³ is substituted aryl, the optional substituents are preferably selected from halo (most preferably fluoro), C₅₋₂₀ aryl, R, OR, SO₂R and COR, where R is C₁₋₇ alkyl.

Z⁴ is preferably N or CR⁴, where R⁴ is H or Q-Y—X

Z⁵ is preferably a direct bond or CH.

Z⁶ is preferably N, S or CH.

### R⁴

When Z², Z³, Z⁵ and Z⁶ all represent CH, and Z⁴ represents CR⁴, it is preferred that R⁴ is Q-Y—X. If at least one of Z², Z³, Z⁵ and Z⁶ is O, N or S, it is preferred that R⁴ is H.

The preferences for NR^{N3}R^{N4} and NR^{N5}R^{N6} are the same as for compounds of formula (IX).

### EXAMPLES

### General Experimental

Commercially available starting materials were purchased from Sigma-Aldrich (Gillingham, Dorset, UK) and Lancaster (Morecambe, Lancashire, UK). Anhydrous DMF, methanol, ethanol, DCM, acetonitrile and pyridine were obtained from Aldrich in SureSealm bottles. Triethylamine was dried by distillation over calcium hydride and stored over potassium hydroxide, under nitrogen. Tetrahydrofuran (THF) was dried by distillation over sodium benzophenone ketyl under an inert atmosphere. All reactions, unless otherwise stated were carried out under an inert atmosphere of nitrogen or argon.

Melting points were measured on a Stuart Scientific melting point apparatus and are uncorrected.

LCMS spectra were recorded using a Micromass Platform LC in combination with a Waters 996 Photodiode Array Detector, a Waters 600 Controller and a Waters 2700 Sample Manager. Separation was achieved on a Waters Symmetry C₁₈ column (4.6×20 nm) or Waters Atlanis C₁₈ column (4.6×50 nm) using isocratic elution with H₂O (A) and MeOH (B) both containing 0.05% formic acid. The gradient used was A:B 95:5 to 5:95 over 5 min.

NMR spectra were recorded on a Bruker Spectrospin AC 300E spectrometer (¹H at 300 MHz, ¹³C at 75 MHz) or JEOL JNM-LA500 spectrometer (¹H at 500 MHz, ¹³C at 125 MHz) with CDCl₃, d₄-MeOH or d₆-DMSO as the solvent. Chemical shifts (δ) are reported in parts per million (ppm) downfield from tetramethylsilane (TMS). Multiplicities are indicated by s (singlet), d (doublet), t (triplet), q (quartet), m (multiplet), br (broad); or combinations thereof. Coupling constants (J) are measured in Hertz (Hz).

IR spectra were recorded on a Bio-Rad FTS 3000MX diamond ATR as a neat sample. Column chromatography was performed using Davisil (40-63 u A) silica gel. Thin-layer chromatography (TLC) was performed using precoated silica gel 60 F₂₅₄ plates with Aluminium backing and was visualised with ultra-violet (UV) light.

HRMS were obtained by EPSRC National Mass Spectrometry Service Centre, Chemistry Department, University of Wales, SA2 8PP Swansea, using MAT900 of MAT95 apparatus.

### Example 1

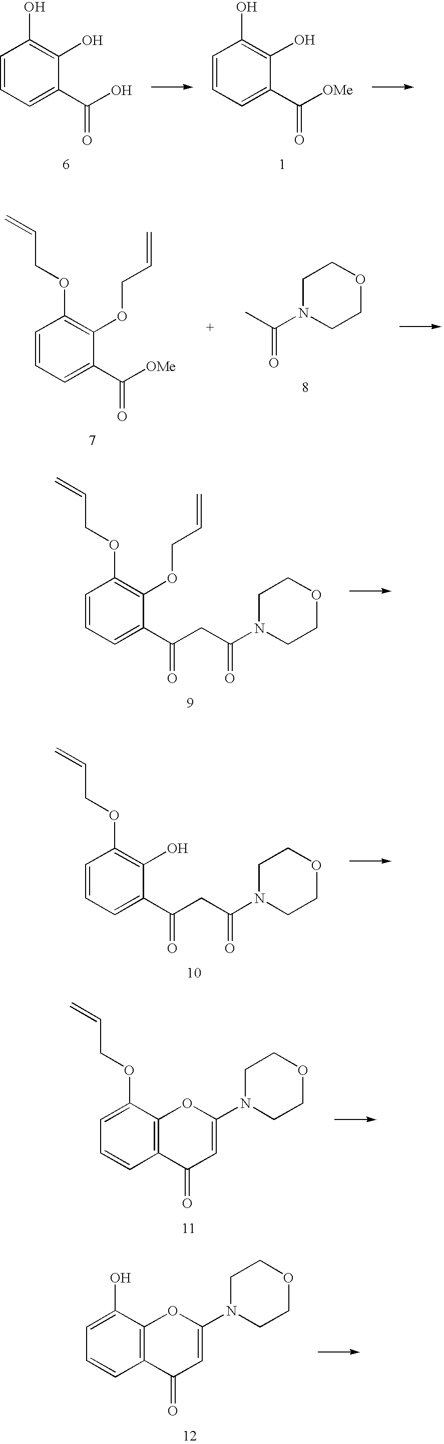

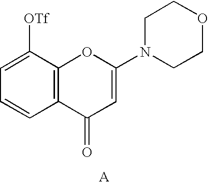

### (a) 2,3-Dihydroxy-benzoic acid methyl ester (1)

This method is disclosed in Coleman, R. S. & Grant, E. B.; J. Am. Chem. Soc., 117(44), 10889 (1995), which is incorporate herein by reference. 2,3 Dihydroxy-benzoic acid (15 g, 97.3 mmol) was dissolved in methanol (150 mL) and cooled to 0° C. with stirring. Concentrated sulfuric acid (9 mL) was added dropwise to the solution. The reaction mixture was heated to reflux for 12 hours and turned brown. The solvent was evaporated, yielding pale brown oil. Ethyl acetate and saturated NaHCO₃ solution were added until effervescence ceased. The aqueous phase was extracted with ethyl acetate (3×150 mL) and the organic layer dried using Na₂SO₄. The resulting solution was concentrated under reduced pressure to give pale brown solid (16.47 g, 99%).

¹H NMR (300 MHz, CDCl₃): δ 3.97 (3H, s), 5.70 (1H, s), 6.82 (1H, t, J=8.0 Hz), 7.10 (1H, d, J=7.9 Hz), 7.32 (1H, d, J=8.0 Hz), 10.9 (1H, s). ¹³C NMR (75 MHz, CDCl₃): δ 52.9, 112.8, 119.6, 120.3, 121.0, 145.4, 149.2, 171.2. m.p.: 83-85° C. I.R.: 3455, 2363, 2222, 2163, 1987, 1668, 1607, 1458, 1340 cm⁻¹. HRMS: [M+NH₄]⁺ calc. 186.0761, meas. 186.0762.

### (b) 2,3-Bis-allyloxy-benzoic acid methyl ester (7)

2,3-Dihydroxy-benzoic acid methyl ester (1)(18.4 g, 109.5 mmol) and potassium carbonate (37.8 g, 273.8 mmol) were dissolved in acetonitrile (180 mL). Allyl bromide (20.6 mL, 241.0 mmol) was added dropwise over 20 minutes to give a pale yellow opaque solution. This was heated to reflux for 9 hours. The opaque yellow liquid formed was diluted with ethyl acetate (150 mL) and washed with water (2×200 mL) and brine (1×150 mL). The resulting orange solution was dried with Na₂SO₄ and concentrated under reduced pressure to give a brown oil (22.32 g, 82%).

¹H NMR (300 MHz, CDCl₃): δ 3.85 (3H, s), 4.55 (4H, d, J=5.4 Hz), 5.10-5.45 (4H, m), 5.85-6.10 (2H, m), 7.08 (2H, d, J=4.8 Hz), 7.17 (1H, t, J=4.8 Hz). ¹³C NMR (75 MHz, CDCl₃): δ 52.2, 69.8, 74.8, 117.7, 118.4, 122.6, 123.7, 125.1, 132.5, 133.2, 133.4, 147.6, 152.8, 166.8. I.R.: 3082, 2952, 2871, 1726, 1581, 1470, 1422, 1357, 1308 cm⁻¹. HRMS: [M+NH₄]⁺ calc. 249.1121, meas. 249.1124.

### (c) 1-(2,3-Bis-allyloxy-phenyl)-3-morpholin-4-yl-propane-1,3-dione (9)

A solution of lithium diisopropilamine (LDA) 1.8 M in THF (2.48 mL, 4.46 mmol), was added over a cooled to −78° C. mixture of N-acetylmorpholine (8)(0.5 mL, 4.46 mmol) into THF (20 mL) dropwise during 30 minutes, maintaining the temperature below −70° C. The reaction mixture was warmed to −10° C., and stirred for 1 hour. A solution of 2,3-bis-allyloxy-benzoic acid methyl ester (7)(0.554 g, 2.23 mmol) in THF (5 mL) was added to the cooled at −78° C. reaction mixture, dropwise and maintaining the temperature below −70° C. The reaction mixture was warmed to room temperature, stirred during 2 h and acidified to pH 1 with aqueous HCl (2 M). The reaction mixture was extracted into DCM (3×30 mL), the combined organic layers were dried with Na₂SO₄ and concentrated. The reaction crude was purified by chromatography on silica with: MeOH:DCM (1:99 to 5:95) as eluent, to give the title compound as a pale yellow oil (0.655 g, 85%).

¹H NMR (300 MHz, CDCl₃): δ 3.20-3.75 (8H, m), 4.15 (2H, s), 4.45 4.65 (4H, m), 5.2-5.4 (4H, m), 5.90-6.10 (2H, m), 7.05-7.25 (3H, m). ¹³C NMR (75 MHz, CDCl₃): δ 40.5, 47.8, 49.6, 67.0, 67.1, 70.0, 74.4, 75.0, 89.5, 117.4, 117.9, 118.3, 119.9, 121.8, 124.5, 129.9, 133.0, 133.4, 133.9, 134.4, 147.0, 147.6, 152.1, 152.5, 166.5, 169.3, 171.7, 196.3. I.R.: 2968, 2916, 2860, 2040, 1671, 1615, 1458 1361, 1268 cm⁻¹. HRMS: [M+H]⁺ calc. 345.1649, meas. 346.1652.

### (d) 1-(3-Allyloxy-2-hydroxy-phenyl)-3-morpholin-4-yl-propane-1,3-dione (10)

A solution of tetrabutylammonium iodide (1.62 g, 4.38 mmol) in DCM (15 mL) was cooled to −78° C. Titanium (IV) chloride (4.40 mL of 1M solution in DCM, 4.38 mmol) was added dropwise over 30 min at −78° C. After 10 minutes 1-(2,3-Bis-allyloxy-phenyl)-3-morpholin-4-yl-propane-1,3-dione (9)(0.72 g, 2.08 mmol) in DCM (15 mL) was added dropwise to give a dark brown solution. The reaction was stirred for 1 hour at −78° C. then allowed to warm to 0° C. over 1 hour. The reaction mixture was poured into saturated aqueous ammonium chloride solution and the aqueous phase extracted in DCM (3×100 mL). The orange organic layer was washed with ammonium chloride solution and dried using Na₂SO₄. This was concentrated under reduced pressure to yield a brown oil, which was purified by column chromatography on silica using MeOH:DCM (2:98) as eluent, to give the product as an oil (0.63 g, 99% yield).

¹H NMR (300 MHz, CDCl₃): δ 3.30-3.50 (8H, m), 4.05 (2H, s), 4.65 (2H, d, J=5.4 Hz), 5.17-5.23 (2H, dd, J_{cis}=10.1 Hz, Jₜᵣₐₙₛ=16.4 Hz), 5.95-6.05 (1H, m), 6.81 (1H, t, J=8.1 Hz), 7.08 (1H, d, J=8.1 Hz), 7.35 (1H, d, J=8.2 Hz). ¹³C NMR (75 MHz, CDCl₃): δ, 42.6, 47.0, 59.3, 66.9, 70.0, 74.3, 119.1, 119.7, 120.2, 121.1, 122.6, 132.9, 147.9, 153.6, 165.4, 201.6. I.R.: 3227, 2966, 2922, 2860, 2247, 1622, 1587, 1444, 1364 cm⁻¹. HRMS: [M+H]⁺ calc. 306.1336, meas. 306.1341.

### (e) 8-Allyloxy-2-morpholin-4-yl-chromen-4-one (11)

1-(3-Allyloxy-2-hydroxy-phenyl)-3-morpholin-4-yl-propane-1,3-dione (10)(0.38 g, 1.25 mmol) was dissolved in DCM (20 mL) and cooled to 0° C. Triflic anhydride (Tf₂O) (0.80 mL, 4.50 mmol) was added with stirring at 0° C. The reaction was warmed to room temperature and stirred during 15 hours. The solvent was evaporated under reduced pressure to give a brown residue. This was redissolved in MeOH (40 mL) and stirred for 1 hour. The solvent was evaporated and then residue diluted with half saturated sodium bicarbonate and the aqueous phase extracted with DCM (3×50 mL). The combined organic layers were washed with brine and dried over Na₂SO₄. The solvent was removed under reduced pressure to yield a yellow solid. This was recrystallised from ethyl acetate and petrol to give a pale cream solid (0.33 g, 92% yield).

¹H NMR (300 MHz, CDCl₃): δ 3.45-3.55 (4H, m), 3.75-3.85 (4H, m), 4.75 (2H, d, J=5.1 Hz), 5.25-5.50 (2H, dd, Jₜᵣₐₙₛ=18.8 Hz, J_{cis}=11.9 Hz), 5.51 (1H, s), 6.10-6.20 (1H, m), 7.12 (1H, d J=8.0 Hz), 7.25 (1H, t, J=7.9 Hz), 7.75 (1H, d, J=7.9 Hz). ¹³C NMR (75 MHz, CDCl₃): δ 45.0, 52.7, 59.2, 67.1, 70.2, 87.6, 115.7, 117.2, 118.3, 124.1, 124.8, 133.0, 144.3, 147.1, 162.8, 177.5. m.p.: 134-136° C. I.R.: 2868, 1641, 1570, 406, 1348, 1269, 1242, 1180, 1112, 1030, 866 cm⁻¹. HRMS: [M+H]⁺ calc. 288.1230, meas. 288.1232.

### (f) 8-Hydroxy-2-morpholine-4-yl-chromen-4-one (12)

To a mixture of 8-Allyloxy-2-morpholin-4-yl-chromen-4-one (11)(0.05 g, 0.174 mmol) in degassed ethanol (4 mL) was added triphenylphosphine ruthenium(I)chloride (11.27 mg, 0.012 mmol) and Dabco (1.95 mg, 0.0174 mmol). This brown mixture was heated under reflux for 3 hours. The reaction mixture was then filtered through a celite pad and concentrated under reduced pressure to give a brown oil. This was purified using column chromatography over silica gel (MeOH: DCM; 10:90) to give a pale yellow solid (0.04 g, 93% yield).

¹H NMR (300 MHz, MeOD): δ 3.75 (4H, m), 3.87 (4H, m), 5.48 (1H, s), 7.12 (2H, m), 7.38 (1H, d, J=7.7 Hz). ¹³C NMR (75 MHz, MeOD): δ 46.5, 55.2, 67.5, 87.4, 116.0, 120.5, 126.7, 131.8, 132.1, 135.5, 135.7, 144.7, 164.7. m.p.: 240-247° C. I.R.: 2966, 2920, 2362, 1718, 1617, 1562, 1480, 1360 cm⁻¹. HRMS: [M+H]⁺ calc. 248.0917, meas. 248.0916.

### (g) Trifluoro-methanesulfonic acid 2-morpholin-4-yl-4-oxo-4H-chromen-8-yl ester (A)

Triethylamine (0.017 mL, 0.11 mmol) was added over a mixture of 8-Hydroxy-2-morpholine-4-yi-chromen-4-one (12)(0.007 g, 0.03 mmol), and N-phenyltriflimide (0.04 g, 0.11 mmol) in THF (4 mL). The reaction mixture was stirred at 70° C. for 4 hours, and at room temperature for 12 hours. Water (10 mL) was added to the reaction mixture, and extracted into DCM (3×10 mL). Combined organic layers were dried over MgSO₄ and concentrated under reduced pressure. Crude reaction mixture was purified by chromatography on column, using MeOH:DCM (2:98 to 5:95), to give the required compound as a pale cream solid (0.006 g, 53%).

¹H NMR (300 MHz, CDCl₃): δ 3.45-3.60 (4H, m), 3.70-3.80 (4H, m), 5.68 (1H, s), 7.50 (1H, t J=8.1 Hz), 7.80 (1H, d J=7.9 Hz), 8.10 (1H, d, J=7.9 Hz) ¹³C NMR (75 MHz, DMSO-d₆): δ 45.1, 65.5, 87.0, 124.9, 125.4, 125.5, 125.6, 136.4, 145.1, 161.8, 173.5. m.p.: 136-138° C. ° C. I.R.: 2866, 1607, 1559, 1483, 1418, 1362 cm⁻¹. HRMS: [M+H]⁺ calc. 280.0410, meas. 280.0411.

The overall yield of compound A was 35%.

### Example 2

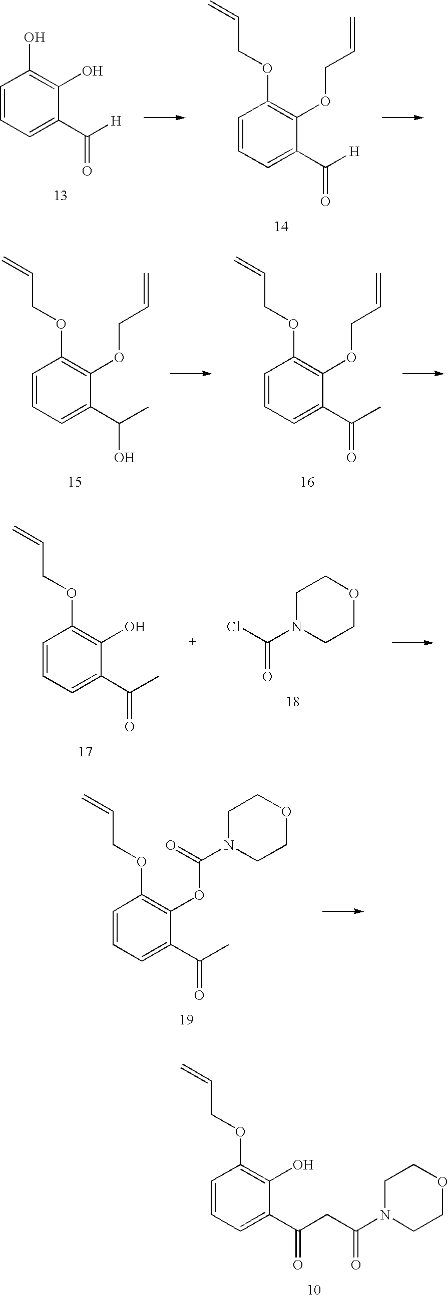

### (a) 2,3-di-allyloxybenzaldehyde (14)

This route is described in Annunziata, R., et al., Eur. J. Org. Chem., 3067 (1999), which is incoroporated herein by reference. To a mixture of 2,3-dihydroxybenzaldehyde (13)(6.22 g, 5 mmol) in acetonitrile (40 mL) with potassium carbonate (15 g, 110 mmol), was added allyl bromide (7.77 mL, 90 mmol) dropwise for 20 minutes, at room temperature and under N₂ atmosphere. The reaction mixture was heated under reflux (80-85° C.) for 4 hours. The mixture was diluted with EtOAc (150 mL) and washed with water (2×200 mL), brine (200 mL), and dried over MgSO₄. The oil product was dried by high vacuum (8.80 g, 89%).

¹H-NMR (300 MHz, CDCl₃): δ 4.50 (4H, d, J=5.5 Hz), 5.25 (4H, m), 6.00 (2H, m), 7.00-7.10 (2H, m), 7.25 (1H, m), 10.30 (1H, s). ¹³C-NMR (300 MHz, CDCl₃): δ 70.23, 75.54, 118.2, 119.3, 119.8, 120.1, 124.5, 130.7, 133.0, 133.5, 151.9, 152.3, 190.8. IR: u 3078, 2865, 1682, 1582, 1465, 1389, 1244, 923 cm⁻¹. HRMS: [M+H]⁺ calc. 219.1016, meas. 219.1015.

### (b) 1-(2,3-diallyloxy-phenyl)-ethanol (15)

To a solution of 2,3-bis-allyloxybenzaldehyde (14)(1 g, 4.5 mmol) in THF (10 ml), was added a solution of methylmagnesium bromide (1.5 mL, 4.5 mmol) in DCM (1.5 mL) for 15 minutes dropwise, at 0° C. and under N₂ atmosphere. The mixture was stirred for 4 hours, and at room temperature for 1 hour. The reaction was quenched with 10% acetic acid solution (25 mL) and ice, extracted in EtOAc (3×50 mL), washed with aqueous sodium metabisulfite solution (2×50 mL), dried over MgSO₄, and concentrated under reduced pressure. The residue was purified by chromatography on silica with EtOAc/petrol (15:85) as eluent, to give the product as colourless oil (0.88 g, 82%).

¹H-NMR (300 MHz, CDCl₃): δ 1.40 (3H, d, J=6.5 Hz), 4.5-4.4 (4H, m), 5.10 (1H, q, J=6.5 Hz), 5.3-5.4 (4H, m), 5.9-6.0 (2H, m), 6.6-6.8 (1H, m), 7.00 (2H, m). ¹³C-NMR (75 MHz. CDCl₃): δ 24.1, 66.3, 69.9, 74.3, 117.8, 118.1, 118.6, 124.5, 130.0, 133.5, 134.5, 139.7, 145.5, 151.7. I.R.: 2992, 2922, 1584, 1471, 1265, 1197, 985, 921, 786 cm⁻¹. HRMS: [M+NH₄]⁺ calc. 252.1594, meas. 252.1598.

### (c) 1-(2,3-diallyloxyphenyl)ethanone (16)

To a solution of 1-(2,3-bisallyloxy-phenyl)-ethanol (15)(5.7 g, 24 mmol) in dry DCM (50 mL), were added Celite (10 g) and PCC (16 g, 73.6 mmol). The reaction mixture was stirred for 5 hours and then filtered through Celite. The filtrated was washed with aqueous HCl (2 M), brine, dried over MgSO₄, and concentrated under reduced pressure. The residue was purified using chromatography on silica with EtOAc/petrol (5:95) as eluent, to give the title compound as colourless oil. (4.86 g, 85%).

¹H-NMR: (300 MHz, CDCl₃); δ 2.55 (3H, s), 4.6-4.5 (4H, m), 5.1-5.3 (4H, m), 5.9-6.1 (2H, m), 7.0-7.2 (3H, m). ¹³C-NMR: (75 MHz. CDCl₃); 631.9, 70.2, 75.0, 117.8, 118.1, 118.7, 121.5, 124.3, 133.2, 133.9, 134.0, 147.0, 152.0, 200.0. I.R.: u 3080, 2867, 1677, 1577, 1463, 1419, 1356, 1307, 1257, 1211 cm⁻¹. HRMS: [M+H]⁺ calc. 233.1172, meas. 233.1172.

### (d) 1-(3-allyloxy-2-hydroxy-phenyl)ethanone (17)

To a solution of nBu₄NI (17 g, 46 mmol) into DCM (15 mL) was added TiCl₄ (46 mL of 1M in DCM, 46 mmol) dropwise for 30 minutes at −78° C. After 10 minutes, a solution of 1-(2,3-diallyloxy-phenyl)-ethanone (16)(4.86 g, 21 mmol) in DCM (20 mL) was added. The reaction was stirred for 4 hours at −78° C. The mixture was poured into an aqueous saturated ammonium chloride solution (100 mL) and extracted into hexane (3×120 mL). The combined organic layers were dried over Na₂SO₄, and filtered. Evaporation of solvent yielded a yellow solid that could be purified by recrystallization from EtOAc to yield the product as yellow needles (3.22 g, 80%).

¹H-NMR: (300 Mz, CDCl₃), δ 2.55 (3H, s), 4.5-4.6 (2H, d J=5.4 Hz), 5.2-5.4 (2H, m), 5.9-6.1 (1H, m), 6.75 (1H, t, J=8 Hz), 7.00 (1H, s), 7.30 (1H, d, J=8 Hz), 12.50 (1H, s). ¹³C-NMR: (75 MHz. CDCl₃); δ 25.9, 69.0, 117.0, 117.07, 118.3, 118.8, 121.3, 131.8, 146.6, 152.2, 203.7. m.p.: 51-52° C. IR: u 2860, 1639, 1448, 1582, 1365, 1321, 1292, 1238, 1031, 935, cm⁻¹. HRMS: [M+H]⁺ calc. 193.0859, meas. 193.0858

### (e) 2-acetyl-6-(allyloxy)phenyl morpholine-4-carboxylate (19)

To a mixture of 1-(3-allyloxy-2-hydroxy-phenyl)ethanone (17)(0.05 g, 0.26 mmol) in acetonitrile (8 mL) with cesium carbonate (0.110 g, 0.34 mmol), was added 4-morpholine carbonyl chloride (18)(0.05 mL, 0.4 mmol) dropwise at room temperature and under N₂ atmosphere. The reaction mixture was heated under reflux (80-85° C.) for 12 hours. The mixture was diluted with EtOAc (30 ml) and washed with water (2×250 ml), brine (30 ml), and dried over MgSO₄. The oil product was purified by chromatography on column using EtOAc/petrol (30:70) as eluent to yield the title product as a pale yellow oil (0.7 g, 86%). ¹H-RMN: (300 MHz, CDCl₃); 2.47 (3H, s), 3.35 (2H, m), 3.60 (6H, m), 4.50 (2H, d, J=5.4 Hz), 5.20-5.30 (2H, dd; J_{cis}=10.1 Hz, Jₜᵣₐₙₛ=16.4 Hz), 5.95-6.05 (1H, m), 7.03 (1H, d, J=8.1 Hz), 7.13 (1H, t, J=8.1 Hz), 7.35 (1H, d, J=8.2 Hz). ¹³C-RMN: (75 MHz, CDCl₃); δ 30.14, 44.6, 45.2, 53.3, 66.5, 69.6, 117.2, 117.6, 121.2, 125.7.1, 132.5, 132.7, 139.7, 151.0, 152.7, 197.7. I.R.: u 2.860, 1718, 1679, 1579, 1415, 1314, 1270, 1197, 1110, 1046, 1012, 853, 787 cm⁻¹. HRMS: [M+H]⁺ calc. 306.1336, meas. 306.1337.

### (f) 1-(3-Allyloxy-2-hydroxy-phenyl)-3-morpholin-4-yl-propane-1,3-dione (10)

To a mixture of 2-acetyl-6-(allyloxy)phenyl morpholine-4-carboxylate (19)(0.08 g, 0.27 mmol) and pyridine (5 mL) was added KOH (0.08 g, 1.3 mmol) as fine power. After 18 hours, the mixture was diluted with 10% acetic acid solution (10 mL), extracted into DCM (3×15 ml) and dried over MgSO₄. The yellow solid was purified by chromatography on silica with EtOAc/petrol (80:20) as eluent to give a brown-yellow oil identified as the required product (0.05 g, 62%).

Compound A was then synthesised from compound (10) as in Example 1, with an overall yield of 19%.

1. A method of synthesising a compound of formula (I): 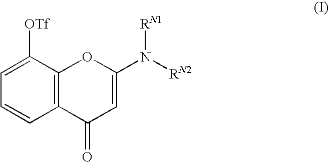
wherein R^{N1} and R^{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
from a compound of formula (III): 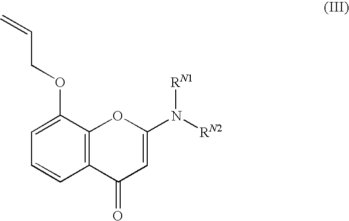
comprising the steps of:
(a) removing the allyl group from the compound of formula (III) with appropriate reaction conditions to yield a compound of formula (II): 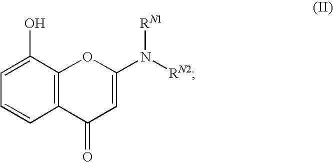
and
(b) reacting the compound of formula (II) with a triflating agent to yield a compound of formula (I).

2. The method of claim 1, wherein in step (a) the removal of the allyl group is carried out using Rh(PPh₃)₃Cl, in the presence of 1,4-diaza-bicyclo[2.2.2]octane in ethanol.

3. The method according to claim 1, wherein step (b) is carried out using triflic anhydride or N-phenyltrifluoromethanesulfonimide (PhNTf₂).

4. The method according to claim 3, wherein step (b) is carried out using PhNTf₂ in triethylamine.

5. The method according to claim 1, wherein the compound of formula (III) is synthesised from a compound of formula (IV): 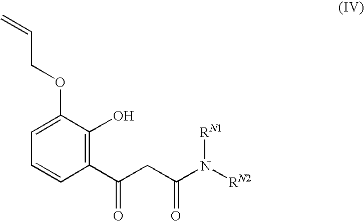
by ring closure.

6. The method according to claim 5, wherein the ring closure is achieved using triflic anhydride in DCM.

7. The method of claim 5, wherein the compound of formula (IV) is synthesised from a compound of formula (V): 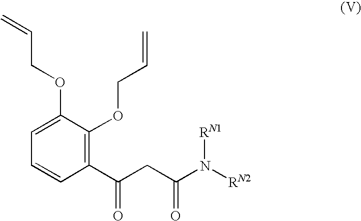
by selective removal of the 2-allyl group.

8. The method of claim 7, wherein the selective removal of the 2-allyl group is carried out using TiCl₄ and Bu₄NI.

9. The method of claim 7, wherein the compound of formula (V) is synthesised by coupling compound 7: 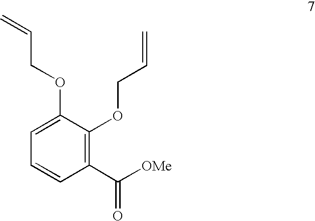
with a compound of formula (VI):

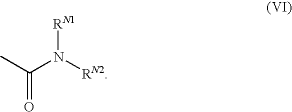

10. The method of claim 9, wherein the coupling is achieved by generating the lithium enolate of the compound of formula (VI) in situ using lithium diisopropylamide (LDA) in THF.

11. The method of claim 9, wherein compound 7 is made from compound 1: 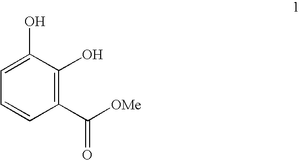
by converting both phenolic groups to allyl ether groups.

12. The method of claim 11, wherein the conversion is carried out using allyl bromide with potassium carbonate in acetonitrile.

13. The method of claim 5, wherein the compound of formula (IV) is synthesised from a compound of formula (VII): 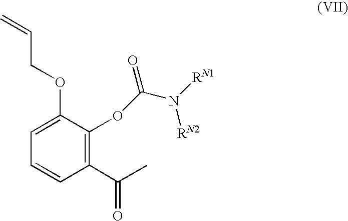
by a Baker-Venkataraman rearrangement.

14. The method of claim 13, wherein the Baker-Venkataraman rearrangement is carried out using potassium hydroxide in pyridine.

15. The method of claim 13, wherein the compound of formula (VII) is synthesised by coupling compound 17: 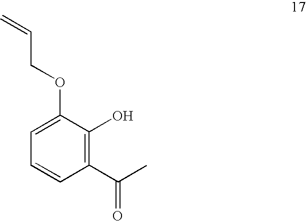
with a compound of formula (VIII):

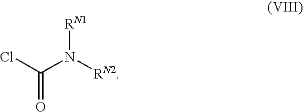

16. The method of claim 15, wherein the coupling is achieved by using cesium carbonate in acetonitrile.

17. The method of claim 15, wherein compound 17 is synthesised from compound 16: 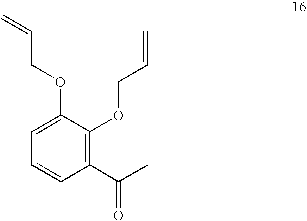
by selective removal of the 2-allyl group.

18. The method of claim 17, wherein the selective removal of the 2-allyl group is carried out using TiCl₄ and Bu₄NI.

19. The method of claim 17, wherein compound 16 is synthesised from compound 15: 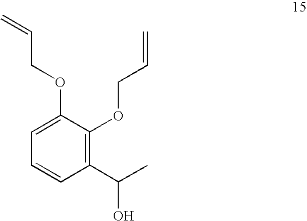
by oxidation.

20. The method of claim 19, wherein the oxidation is carried out using pyridinium chlorochromate (PCC), MnO₂ or the Dess-Martin reagent.

21. The method of claim 20, wherein the oxidation is carried out using PCC.

22. The method of claim 19, wherein compound 15 is synthesised from compound 14: 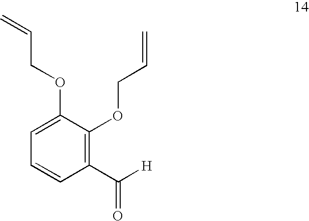
by methylation by use of a Grignard reagent.

23. The method of claim 22, wherein the methylation is achieved by treatment with MeMgBr.

24. The method of claim 21, wherein compound 14 is synthesised from compound 5: 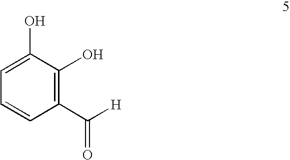
by conversion of both phenolic groups to allyl ether groups.

25. The method of claim 24, wherein the conversion is carried out using allyl bromide with potassium carbonate in acetonitrile.

26. The method of claim 1, wherein the compound of formula (I) is further converted to a compound of formula (IX): 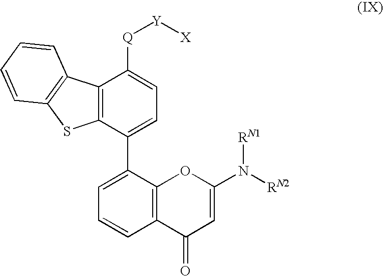
wherein:
R^{N1} and R^{N2} are as defined for compound (I);
Q is —NH—C(═O)— or —O—;
Y is an optionally substituted C₁₋₅ alkylene group;
X is selected from SR^{S1} or NR^{N3}R^{N4}, wherein,
R^{S1}, or R^{N3} and R^{N4} are independently selected from hydrogen, optionally substituted C₁₋₇ alkyl, C₅₋₂₀ aryl, or C₃₋₂₀ heterocyclyl groups, or R⁴ and R⁵ may together form, along with the nitrogen atom to which they are attached, an optionally substituted heterocyclic ring having from 4 to 8 ring atoms;
if Q is —O—, X is additionally selected from —C(═O)—NR^{N5}R_{N}, wherein R^{N5} and R^{N6} are independently selected from hydrogen, optionally substituted C₁₋₇ alkyl, C₅₋₂₀ aryl, or C₃₋₂₀ heterocyclyl groups, or R^{N5} and R^{N6} may together form, along with the nitrogen atom to which they are attached, an optionally substituted heterocyclic ring having from 4 to 8 ring atoms; and
if Q is —NH—C(═O)—, —Y—X may additionally selected from C₁₋₇ alkyl.
